(19) United States
(12) Patent Application Publication
(10) Pub. No.: US 2023/0158212 A1
(43) Pub. Date: May 25, 2023

(54) COMPOSITE SCAFFOLD MATERIAL

(71) Applicant: Nanyang Technological University, Singapore (SG)

(72) Inventors: Chor Yong TAY, Singapore (SG); Jun Kit WANG, Singapore (SG); Cigdem CIMENOGLU, Singapore (SG)

(21) Appl. No.: 17/912,707

(22) PCT Filed: Apr. 16, 2021
(86) PCT No.: PCT/SG2021/050219

§ 371 Date: Sep. 19, 2022

(30) Foreign Application Priority Data
Apr. 16, 2020 (SG) 10202003503Y

(51) Int. Cl. A61L 27/56; A61L 27/46; C08L 89/06; C12N 5/00

(52) U.S. Cl. CPC A61L 27/56; A61L 27/46; C08L 89/06; C12N 5/0068; A61L 2430/02; C12N 2533/54; C12N 2537/10

(57) ABSTRACT

Disclosed herein is a composite scaffold material that includes a crosslinked polymer matrix formed from a non-mammalian collagen and a crosslinking agent and/or a crosslinked polymer matrix formed from a non-mammalian collagen that has undergone self-crosslinking, and a plurality of calcium phosphate particles distributed within the crosslinked polymer matrix, where the composite scaffold material is porous. Also disclosed herein are methods of manufacturing the composite scaffold material and uses thereof. Further disclosed herein is a method of obtaining collagen from a non-mammalian source.

### FIELD OF INVENTION

This invention relates to the use of non-mammalian (e.g. bullfrog, fish, etc) skin-derived collagen, calcium phosphate, materials comprising thereof, and uses of the aforementioned materials.

### BACKGROUND

The listing or discussion of a prior-published document in this specification should not necessarily be taken as an acknowledgement that the document is part of the state of the art or is common general knowledge.

In recent years, aquaculture has been one of the fastest-growing food production sectors due to demand from rapid global population growth. However, the increase in food production has resulted in a substantial increase in waste and by-product generation. Notably, only 30-50% of the biomass of the farmed species produced by aquafarming are used for direct human consumption. Aquaculture side-streams that get recycled are currently used for low-value purposes, such as animal feed or in composting materials which are of low economic value (J.-K. Kim, Fish. Aquat. Sci. 2011, 14, 230-233; and I. S. Arvanitoyannis et al., Int. J. Food Sci. Technol. 2008, 43, 726-745), while by-products such as fins, scales, skins and viscera are discarded in a quantity that exceeds 20 million tons per year (M. Govindharaj et al., J. Clean. Prod. 2019, 230, 412-419; and G. Caruso, J. FisheriesSciences.com 2015, 9, 80-83). Although various preventive measures have been taken to reduce the production of waste, the majority of the waste is either disposed by landfill or incineration, which are environmentally unfriendly and economically unwise.

To maximize resource reuse and minimize waste generation, valorisation efforts need to be augmented with the aim to produce high-value products. The vast amount of aquaculture by-products actually contain numerous valuable bioactive compounds such as peptides, collagen, lipids, and calcium phosphates, which could potentially be exploited for high-value biomedical applications (P. Ideia et al., Waste Biomass Valor. 2020, 11, 3223-3246; and C. Bai et al., ACS Sustain. Chem. Eng. 2017, 5, 7220-7227). Therefore, efforts to reduce and valorise waste generated from aquaculture-related activities for the production of high-quality biomaterials have gained significant traction in recent years (G. K. Pal et al., Innov. Food Sci. Emerg. Technol. 2016, 37, 201-215; and C. Xu et al., Chem. Soc. Rev. 2019, 48, 4791-4822), and the “waste-to-resource” paradigm has emerged as an attractive approach to augment efforts in waste management (K. L. Ong et al., Bioresour. Technol. 2018, 248, 100-112; and Z. Wu et al., Environ. Sci. Technol. 2020, 54, 9681-9692).

Bone is composed of two pre-dominant components, namely type I collagen (organic phase) and hydroxyapatite (HA) (inorganic phase). In general, an ideal scaffold material for bone tissue engineering should be:

(i) intrinsically non-toxic to ensure good biocompatibility;
(ii) compatible with processing techniques that would enable emulation of the biophysical and biochemical complexity of the host tissue;
(iii) porous to optimize cell infiltration and exchange of nutrients/metabolic wastes;
(iv) of adequate mechanical strength under physiological loading conditions;
(v) mechanically and structurally stable to withstand loading; and
(vi) osteoconductive or osteoinductive to support “restitutio ad integrum” recovery.

See for example T. Ghassemi et al., Arch. Bone Jt. Surg. 2018, 6, 90; K. Glenske et al., Int. J. Mol. Sci. 2018, 19, 826; F. Baino. (2017); Scaffolds in Tissue Engineering: Materials, Technologies and Clinical Applications. BoD—Books on Demand; and X. Zhang et al., ACS Sustain. Chem. Eng. 2020, 8, 2106-2114.

One waste-to-resource approach taken for the production of resource-efficient bioactive materials is the harvesting of natural HA from biowaste, such as eggshells, fish scales, and mussel shells targeted for bone tissue engineering (S.-L. Bee et al., Ceram. Int. 2020, 46, 17149-17175; K. Ronan et al., ACS Sustain. Chem. Eng. 2017, 5, 2237-2245; and Y. Y. Chun et al., Macromol. Biosci. 2016, 16, 276-287). Another well-known research direction is the transformation of fish waste, such as fish skins and scales into collagen for soft tissue engineering, including skin and lymphatic vessel regeneration (J. K. Wang et al., Acta Biomater. 2017, 63, 246-260; and A. Afifah et al., IOP Conf. Ser.: Earth Environ. Sci. 2019, 335, 012031). However, current collagen extraction methods have poor yield and a long processing time. In addition, consumers often refuse to use products that are produced from bovine collagen due to potential risks of infectious diseases (C. H. Theng et al., Int. J. Adv. Sci. Eng. Inf. Technol. 2018, 8, 832-841).

Therefore, there is a need to find new methods to convert aquafarming waste into high-value products that are suitable for use in a variety of applications. These applications may include, for example, tissue engineering products and the like, amongst others.

### SUMMARY OF INVENTION

It has been surprisingly found that it is possible to turn aquafarming waste products into high-value materials suitable for use in tissue engineering. In particular, it has been surprisingly found that it is possible to generate a composite scaffold material with superior properties from a combination of non-mammalian skin-derived collagen (e.g. from bullfrog skin) and calcium phosphate particles. The associated methods of generating collagen and calcium phosphate from aquaculture waste products makes the scaffold both environmentally friendly and economically feasible.

1. A composite scaffold material comprising:
one or both of a crosslinked polymer matrix formed from a non-mammalian collagen and a crosslinking agent and a crosslinked polymer matrix formed from a non-mammalian collagen that has undergone self-crosslinking; and
a plurality of calcium phosphate particles distributed within the crosslinked polymer matrix, wherein the composite scaffold material is porous.

2. The composite scaffold material according to Clause 1, wherein the crosslinking agent is a pharmaceutically acceptable crosslinking agent.

3. The composite scaffold material according to Clause 1 or Clause 2, wherein the crosslinking agent is selected from one or more of the group consisting of genipin and compounds comprising two or more crosslinkable functional groups selected from the group consisting of amino, carboxylic acid, ester, aldehyde and epoxide functional groups (e.g. the crosslinking agent is selected from compounds comprising two or more crosslinkable functional groups selected from the group consisting of aldehyde and epoxide functional groups).

4. The composite scaffold material according to any one of the preceding clauses, wherein the crosslinking agent is selected from compounds comprising two crosslinkable functional groups, optionally wherein the crosslinking agent is selected from one or more of the group consisting of glutaraldehyde and 1,4-butanediol diglycidyl ether (e.g. the crosslinking agent is 1,4-butanediol diglycidyl ether).

5. The composite scaffold material according to any one of the preceding clauses, wherein when the crosslinked polymer matrix is formed from a non-mammalian collagen that has undergone self-crosslinking, the non-mammalian collagen has been crosslinked by a transglutaminase.

6. The composite scaffold material according to any one of the preceding clauses, wherein, when present, the crosslinking agent forms from 3 to 15 wt %, such as from 8 to 10 wt % of the crosslinked polymer matrix formed from a non-mammalian collagen and a crosslinking agent.

7. The composite scaffold material according to any one of the preceding clauses, wherein the calcium phosphate particles have a diameter of from 10 nm to 20 μm, such as from 50 nm to 10 μm, such as from 500 nm to 3 μm, optionally wherein the calcium phosphate particles have a diameter of from 0.5 to 20 μm, such as from 1 to 10 μm, such as from 1.5 to 3 μm, such as about 1.6 μm.

8. The composite scaffold material according to any one of the preceding clauses, wherein the calcium phosphate particles are hydroxyapatite, optionally wherein the hydroxyapatite is monophase hydroxyapatite.

9. The composite scaffold material according to Clause 8, wherein the hydroxyapatite is derived from fish scales, optionally wherein the fish scales are from snakehead fish and/or elasmoid scales derived from teleosts.

10. The composite scaffold material according to any one of the preceding clauses, wherein the non-mammalian collagen is type I collagen.

11. The composite scaffold material according to any one of the preceding clauses, wherein the non-mammalian collagen is derived from bullfrog skin, optionally wherein the bullfrogs belong to the genus rana (e.g. the bullfrogs are of the species Rana catesbeiana).

12. The composite scaffold material according to any one of the preceding Clauses, wherein the composite scaffold material has a porosity of from 90 to 99%, such as from 93 to 98.5%, such as 95 to 98%.

13. The composite scaffold material according to any one of the preceding clauses, wherein one or more of the following apply:

(ai) the composite scaffold material has a compression modulus of from 0.5 to 4.5 kPa, such as from 1.0 to 4.2 kPa, such as from 1.9 to 4 kPa, such as from 2.5 to 4 kPa, such as from 3 to 3.5 kPa;
(aii) the composite scaffold material is further coated with calcium phosphate particles, optionally wherein the calcium phosphate is hydroxyapatite (e.g. the hydroxyapatite is monophase hydroxyapatite); and
(aiii) more than 50% of the composite scaffold material has degraded after 8 days when subjected to a 1× phosphate-buffered saline, and the degradation is measured by bicinchoninic acid (BCA) protein assay kit.

14. Use of a composite scaffold material according to any one of Clauses 1 to 13 in the manufacture of a medicament for use in tissue engineering in a subject in need thereof.

15. A composite scaffold material according to any one of Clauses 1 to 13 for use in tissue engineering in a subject in need thereof.

16. A method of tissue engineering comprising the step of providing a suitable amount of a composite scaffold material according to any one of Clauses 1 to 13 to a subject in need thereof.

17. A method of in vitro tissue engineering, wherein the method comprises the steps of:

(bi) supplying a composite scaffold material according to any one of Clauses 1 to 13;
(bii) adding cells and a suitable cellular nutrient mixture to the composite scaffold material; and
(biii) allowing the cells to grow on the composite scaffold material for a period of time.

18. The use according to Clause 14, the composite scaffold material for use according to Clause 15, the method according to Clause 16 and the method according to Clause 17, wherein the tissue engineering is bone tissue engineering.

19. A method of providing a collagen precursor mixture from a non-mammalian source, the method comprising the steps of:

(a) providing a mixture of pre-treated skins from a non-mammalian animal in an acidic solvent; and
(b) subjecting the mixture to mechanical blending to provide the collagen precursor mixture in the form of a paste.

20. The method according to Clause 19, wherein one or more of the following apply:

(ci) the acidic solvent is aqueous acetic acid, optionally wherein the aqueous acetic acid has a molarity of from 0.1 to 1 M, such as from 0.3 to 0.7 M, such as about 0.5 M;
(cii) the acidic solvent is provided in a weight to volume ratio of from 0.1:10 to 2:10, such as 1:10, where the weight refers to the weight of the pre-treated skins from a non-mammalian animal and the volume refers to the volume of the acidic solvent;
(ciii) the blending is conducted over a period of from 1 to 20 minutes, such as from 2 to 10 minutes, such as about 5 minutes; and
(civ) the blending is conducted at from 20,000 to 50,000 rpm, such as from 30,000 to 40,000 rpm, such as about 35,000 rpm;
(cv) the entire method is conducted at a temperature of from 0.1 to 10° C., such as from 1 to 5° C., such as about 4° C.;
(cvi) the pre-treated skins are bullfrog skin, optionally wherein the bullfrogs belong to the genus Rana (e.g. the bullfrogs are of the species Rana catesbeiana).

21. A method of providing collagen from a non-mammalian source, the method comprising the steps of:

(aa) providing a collagen precursor mixture in the form of a paste;
(ab) diluting the paste with water and centrifuging the resulting diluted paste to provide a collagen solution and a pellet comprising pigments and collecting the collagen solution;
(ac) adding an inorganic salt to the collagen solution for a period of time (e.g. from 12 to 48 hours, such as from 18 to 24 hours (e.g. from 12 to 18 hours)) to precipitate out a collagen salt, which is then collected by centrifugation;
(ad) adding an acidic solvent to the collected collagen salt to provide a free collagen mixture and subjecting the free collagen mixture to dialysis to provide a solution of collagen from a non-mammalian source.

22. The method according to Clause 21, wherein one or more of the following apply:

(ba) the collagen precursor mixture is obtained using the method according to Clause 19;
(bb) the paste is diluted by water in a ratio of from 1:10 to 1:30 vol/vol, such as from 1:10 to 1:20 vol/vol, such as about 1:10 vol/vol;
(bc) the centrifugation in step (ab) of Clause 21 is conducted at from 15,000 to 50,000×g, such as from 20,000 to 35,000×g, such as about 25,000×g;
(bd) the centrifugation in step (ab) of Clause 21 is conducted for a period of from 5 to 45 minutes, such as from 10 to 30 minutes, such as about 15 minutes;
(be) the inorganic salt in step (ac) of Clause 21 is selected from one or more of sodium sulphate, ammonium sulphate, potassium chloride, and sodium chloride (e.g. the inorganic salt is sodium chloride), optionally wherein the inorganic salt is provided as an aqueous solution having a concentration of from 0.5 to 4.0 M, such as from 0.5 to 1.5 M, such as about 0.9 M;
(bf) the centrifugation in step (ac) of Clause 21 is conducted at from 3,000 to 10,000×g, such as from 4,000 to 6,000×g, such as about 5,500×g;
(bg) the centrifugation in step (ac) of Clause 21 is conducted for a period of from 5 to 45 minutes, such as from 10 to 30 minutes, such as about 15 minutes;
(bh) the acidic solvent in step (ad) of Clause 21 is aqueous acetic acid, optionally wherein the aqueous acetic acid has a molarity of from 0.1 to 1 M, such as from 0.3 to 0.7 M, such as about 0.5 M;
(bi) the dialysis in step (ad) of Clause 21 is conducted in two rounds, wherein:
(i) the first round dialysis makes use of aqueous acetic acid at a concentration of from 0.01 to 0.3 M, such as from 0.05 to 0.2 M, such as about 0.1 M; and
(ii) the second round dialysis makes use of water;
   (bj) the entire method is conducted at a temperature of from 0.1 to 10° C., such as from 1 to 5° C., such as about 4° C.;
   (bk) step (ac) of Clause 21 is only conducted once.

23. The method according to Clause 21 or Clause 22, wherein the solution of collagen from a non-mammalian source is lyophilised.

24. A method of providing a composite scaffold material according to any one of Clauses 1 to 13, wherein the method comprises the steps of:

(di) providing a solution of a non-mammalian collagen;
(dii) adding calcium phosphate particles and one or both of a crosslinking agent and an agent to promote self-crosslinking to the solution of a non-mammalian collagen to form a reaction mixture and allowing the reaction mixture to react for a period of time; and
(diii) removing the solvent to provide the composite scaffold material.

25. The method according to Clause 24, wherein:
after the period of time, the reaction mixture of step (dii) is deposited onto a flat substrate and allowed to dry to provide the composite scaffold material in the form of a film; or
after the period of time, the reaction mixture of step (dii) is deposited into a mold and lyophilised to provide the composite scaffold material in the form of a three-dimensional structure.

26. The method according to Clause 24 or Clause 25, wherein one or more of the following apply:

(ca) the non-mammalian collagen in the solution of a non-mammalian collagen is provided at a concentration of from 5 to 20 mg/mL, such as about 10 mg/mL;
(cb) the solvent in the solution of a non-mammalian collagen is aqueous acetic acid, optionally wherein the aqueous acetic acid has a molarity of from 0.1 to 1 M, such as from 0.3 to 0.7 M, such as about 0.5 M;
(cc) the calcium phosphate particles are provided in a weight to weight ratio relative to collagen of from 0.5:1 to 2:1, such as about 1:1;
(cd) the crosslinking agent, when present, is provided in an amount of from 3 to 15 wt %, such as from 8 to 10 wt % relative to the dry weight of the collagen;
(ce) the period of time is from 12 to 48 hours, such as from 18 to 32 hours, such as about 24 hours.

### DRAWINGS

FIG. 1 depicts a schematic diagram of the extraction process employed to recover collagen from bullfrog skins.

FIG. 2 depicts the characterization of BFCol: (A) digital picture; (B) ATR-FTIR profile; (C) SDS-PAGE image; (D) AFM images of BFCol and Bovine Collagen; and (E) AFM image of BFCol nanofibrils (Scale bar=1 cm for digital picture; 500 nm for AFM images).

FIG. 3 depicts a schematic diagram of the processes involved in the isolation of hydroxyapatite (HA) from fish scales.

FIG. 4 depicts the characterization of fish scale derived HA: (A) Pictograph of the HA. Scale bar=1 cm; (B) ATR-FTIR spectrum of the HA; (C) XRD profile of the monophase HA; and (D) DLS analysis showing the average particle size of the isolated HA.

FIG. 5 depicts the fabrication and characterization of BDDE-crosslinked BFCol/HA (B-BFCol/HA) hybrid biocomposite: (A) ATR-FTIR spectra confirming the BDDE crosslinking and the presence of HA in the hybrid biocomposite scaffold, and a digital picture of the B-BFCol/HA hybrid biocomposite. Scale bar=1 cm; (B) Cross-sectional SEM images showing the multi-scale morphology of the B-BFCol/HA hybrid biocomposite (i.e., micro-roughness of HA and nano-roughness of BDDE-crosslinked (B-BFCol)). Scale bar=100 μm (×200), 1 μm (×15,000), and 1 nm (×100,000); (C) Porosity measurement of B-BFCol/HA hybrid biocomposite; (D) Mechanical analysis of the hybrid biocomposite scaffold. *denotes statistical difference between indicated experimental groups at p<0.05; and (E) degradation profile.

FIG. 6 depicts the absorbency of B-BFCol/HA hybrid composite.

FIG. 7 depicts the cytocompatibility analyses of B-BFCol/HA hybrid biocomposite: (A) Expression level of inflammatory mRNA transcripts in macrophage exposed to B-BFCol/HA hybrid biocomposite; (B) Quantitative measurement of the hFOB 1.19 growth in B-BFCol/HA hybrid biocomposite over 7 days of culture; and (C) Cross-sectional microscopic images of the hFOB 1.19-seeded hybrid biocomposite. The cell nucleus (blue) was counter-stained with DAPI dye. Scale bar=5 mm (pictograph) and 100 μm (microscopic image). *denotes statistical difference between indicated experimental groups at p<0.05.

FIG. 8 depicts the osteogenic studies of B-BFCol/HA hybrid biocomposite: (A) Expression of early osteoblast marker (ALPL) in hFOB 1.19; (B) Expression of late osteoblast marker (BGLAP) in hFOB 1.19; (C) Immunocytochemical staining images of hFOB 1.19 counter-stained for cell nucleus (blue) and osteocalcin (green). Scale bar=100 μm; and (D) Alizarin red S (red) staining of cell-seeded hybrid biocomposite. Scale bar=200 μm * denotes statistical difference between indicated experimental groups at p<0.05.

FIG. 9 depicts the timeline for the different collagen extraction methods: (a) new collagen extraction method; and (b) traditional acid solubilization method.

### DESCRIPTION

Thus, in a first aspect of the invention, there is provided a composite scaffold material comprising:
one or both of a crosslinked polymer matrix formed from a non-mammalian collagen and a crosslinking agent and a crosslinked polymer matrix formed from a non-mammalian collagen that has undergone self-crosslinking; and
a plurality of calcium phosphate particles distributed within the crosslinked polymer matrix, wherein the composite scaffold material is porous.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As will be appreciated, the crosslinked polymer matrix may be formed from:
a non-mammalian collagen and a crosslinking agent;
a non-mammalian collagen that has undergone self-crosslinking; or
a combination of both.

Herein all of these possibilities may be referred to as the crosslinked polymer matrix and this term should, unless otherwise specified, be understood accordingly.

The term “crosslinking agent” when used herein refers to a compound that can form at least two covalent bonds with the collagen, whether by inherently having at least two functional groups that can crosslink to the collagen or through reaction with itself to generate at least two functional groups capable of crosslinking. Any suitable crosslinking agent may be used. However, as the materials discussed herein may be used in implants in the human or animal body, the crosslinking agent may be a pharmaceutically acceptable crosslinking agent. For example, the crosslinking agent may be selected from one or more of the group consisting of genipin and compounds comprising two or more (e.g. 2, 3, 4, 5, 6, or 7) crosslinkable functional groups selected from the group consisting of amino, carboxylic acid, ester, aldehyde and epoxide functional groups. For example, the crosslinking agent may be selected from compounds comprising two or more crosslinkable functional groups selected from the group consisting of aldehyde and epoxide functional groups. Particular crosslinking agents that may be mentioned herein may have two crosslinkable functional groups.

Examples of suitable crosslinking agents include, but are not limited to glutaraldehyde, genipin, 1,4-butanediol diglycidyl ether (e.g. the crosslinking agent is 1,4-butanediol diglycidyl ether) and combinations thereof. Examples of suitable crosslinking agents with two crosslinkable functional groups include, but are not limited to, glutaraldehyde and 1,4-butanediol diglycidyl ether (e.g. the crosslinking agent is 1,4-butanediol diglycidyl ether).

When present, the crosslinking agent may be used in any suitable amount of the crosslinked polymer matrix. It will be understood that the crosslinking agent will be covalently bonded to collagen. For example, the (covalently bonded) crosslinking agent may form from 3 to 15 wt %, such as from 8 to 10 wt % of the crosslinked polymer matrix formed from a non-mammalian collagen and a crosslinking agent.

As noted above, the crosslinked polymer matrix may have undergone self-crosslinking. This may be achieved through the use of individual chemical reactions performed by a skilled person to affect self-crosslinking or, more particularly, it may be achieved by the use of one or more enzymes to affect the crosslinking. For example, the self-crosslinking may be achieved by the use of a transglutaminase. It will be appreciated that a self-crosslinked polymer matrix may not substantially contain the chemicals/enzyme(s) used to affect the self-crosslinking. This is because these substances may be washed away during the processing of the crosslinked polymer matrix.

Any suitable calcium phosphate particles may be used in the composite scaffold materials disclosed herein. For example, the calcium phosphate particles may have a diameter of from 10 nm to 20 μm, such as from 50 nm to 10 μm, such as from 500 nm to 3 μm. In more particular embodiments that may be mentioned herein, the calcium phosphate particles may have a diameter of from 0.5 to 20 μm, such as from 1 to 10 μm, such as from 1.5 to 3 μm, such as about 1.6 μm. Without wishing to be bound by theory, it is believed that the use of calcium phosphate particles having a nanometer size (e.g. from 10 to 500 nm, such as from 50 to 100 nm) may enhance the mechanical properties compared to the use of micron-sized particles. For example, the use of nano-sized calcium phosphate (e.g. hydroxyapatite) particles may enhance the matrix stiffness (e.g. up to about a 6.2-fold increase in compressive modulus relative to the crosslinked collagen alone used herein). This may be beneficial in certain cases, but may not be desired or necessary in all applications that the materials disclosed herein may be used for, as such, the micron-sized particles may be suitable for all of the possible applications mentioned herein.

While any suitable form of calcium phosphate may be used, in particular embodiments of the invention that may be mentioned herein, the calcium phosphate particles may be in the form of hydroxyapatite. In yet more particular embodiments of the invention that may be mentioned herein, the hydroxyapatite may be in the form of monophase hydroxyapatite.

The calcium phosphate and, more particularly, hydroxyapatite may be obtained from any suitable source. This may be from a mineral source, through synthetic means from inorganic starting materials or from a natural source. In embodiments of the invention that may be mentioned herein, the calcium phosphate may be derived from fish scales, which may function as a source of hydroxyapatite. Any suitable fish scales may be used. For example, the fish scales may be from snakehead fish and/or elasmoid scales derived from teleosts.

As mentioned hereinbefore, only 30-50% of aquafarming production is used for direct human consumption. By-products such as fins, scales, skins, and viscera are discarded in a quantity that exceeds 20 million tons per year, with the majority either disposed by landfill or incineration. Thus, as will be appreciated, the use of fish scales to generate hydroxyapatite may help to significantly reduce food waste and make better economic use of the valuable resources found in the by-products of fish.

In embodiments of the invention, the composite scaffold material may use any suitable non-mammalian collagen. A particular non-mammalian collagen that may be used herein is type I collagen.

Any suitable non-mammalian species may be used to generate the collagen (e.g. type I collagen) used in the composite scaffold materials disclosed herein. Such species include vertebrates and invertebrates. More particularly, the non-mammalian collagen may be derived from bullfrog skin, optionally wherein the bullfrogs belong to the genus rana (e.g. the bullfrogs are of the species Rana catesbeiana).

Again, for the reasons discussed above, it is noted that the use of bullfrog skins to produce type I collagen enables the capture of a resource that may have otherwise been wasted. In addition, it is also noted that the use of collagen derived from non-mammalian resources may reduce or eliminate risks associated with disease from mammalian sources (e.g. bovine spongiform encephalopathy (BSE), transmissible spongiform encephalopathy (TSE) and foot-and-mouth disease (FMD). Therefore, non-mammalian sources of collagen (e.g. bullfrog collagen) can be regarded as a safer source of collagen as compared to those from mammalian species.

As mentioned herein, the composite scaffold material is porous. This porosity may be beneficial when the material is used in an implant as it allows for the ingress of body fluids, cells etc., as well as allowing for the composite scaffold material to undergo a faster degradation than would otherwise be the case. While any suitable level of porosity may be used, in particular embodiments of the invention that may be mentioned herein, the composite scaffold material may have a porosity of from 90 to 99%, such as from 93 to 98.5%, such as 95 to 98%. Details of how to measure the porosity of the composite scaffold materials mentioned herein are provided in the examples section below.

The composite scaffold material may have a compression modulus of from 0.5 to 4.5 kPa, such as from 1.0 to 4.2 kPa, such as from 1.9 to 4 kPa, such as from 2.5 to 4 kPa, such as from 3 to 3.5 kPa. This may refer to a material made using hydroxyapatite particles in the micron diameter range. Without wishing to be bound by theory, this compression modulus may be increased if needed or desired through the incorporation of nano-sized calcium phosphate (e.g. hydroxyapatite) particles, either in place of or in addition to the micron-sized particles. The inclusion of nano-sized calcium phosphate particles may increase the compression modulus values by around 6-fold (e.g. around 6.2-fold) relative to the crosslinked collagen material's native compressive modulus. Additionally or alternatively, the compression modulus may also be increased if needed or desired through coating the composite scaffold material with calcium phosphate particles. This coating of calcium phosphate particles may increase the compression modulus values by around 26-fold (e.g. around 26.2-fold) relative to the crosslinked collagen material's native compressive modulus. As such, it is possible to tailor the compressive modulus to match the desired application of the composite scaffold material by the use of the three options mentioned herein. That is, micron-sized calcium phosphate particles, nano-sized calcium phosphate particles and coating the composite scaffold material with calcium phosphate particles. For the avoidance of doubt, any calcium phosphate particles may be used in these embodiments, but in particular embodiments that may be mentioned herein, the calcium phosphate may be hydroxyapatite (e.g. in the form of monophase hydroxyapatite).

The composite scaffold materials may be particularly useful in applications concerning tissue engineering and particularly bone tissue engineering. As such, it is advantageous that the composite scaffold materials disclosed herein are capable of degrading over time in order to allow for new tissue to form and occupy the space vacated by the degrading composite scaffold material. As such, in embodiments of the invention that may be mentioned herein, the composite scaffold material may be one in which more than 50% of the composite scaffold material has degraded after 8 days when subjected to a 1×phosphate-buffered saline, and the degradation is measured by bicinchoninic acid (BCA) protein assay kit.

As intimated above, the composite scaffold material may be useful in tissue engineering. Thus, in a further aspect of the invention, there is provided:
(a) use of a composite scaffold material as described herein in the manufacture of a medicament for use in tissue engineering in a subject in need thereof;
(b) a composite scaffold material as described herein for use in tissue engineering in a subject in need thereof; and
(c) a method of tissue engineering comprising the step of providing a suitable amount of a composite scaffold material as described herein to a subject in need thereof.

As will be appreciated, while the composite scaffold material may be useful in tissue engineering applications in vivo, it may also be useful for applications in vitro. As such, a further aspect of the invention provides a method of in vitro tissue engineering, wherein the method comprises the steps of:

(bi) supplying a composite scaffold material as described herein;
(bii) adding cells and a suitable cellular nutrient mixture to the composite scaffold material; and
(biii) allowing the cells to grow on the composite scaffold material for a period of time.

Examples of such applications may include disease modelling, or for drug screening purposes, such as to identify drugs for bone cancer/bone metastasis.

As will be appreciated, the above methods may relate to any type of tissue engineering where the composite scaffold material is suitable. In particular embodiments of the invention, the various uses and methods above may relate to bone tissue engineering.

As mentioned hereinbefore, the collagen used in the composite scaffold materials above may be derived from a non-mammalian source. It has been surprisingly found that it is possible to generate substantial quantities of collagen through a revised method. Thus, in a further aspect of the invention, there is disclosed a method of providing a collagen precursor mixture from a non-mammalian source, the method comprising the steps of:

(a) providing a mixture of pre-treated skins from a non-mammalian animal in an acidic solvent; and
(b) subjecting the mixture to mechanical blending to provide the collagen precursor mixture in the form of a paste.

In particular, it has surprisingly been found that subjecting skins to mechanical blending enables not only the yield of collagen to be significantly increased compared to conventional methods, but also a substantial reduction in the processing time required. That is, compared to the commonly used acid solubilization methods for collagen extraction, this new method is much simpler and faster as it can be completed within approximately 11 days (FIG. 8a). Without wishing to be bound by theory, it is believed that the incorporation of the blending step, which cuts the skin into extremely small pieces, facilitates the isolation of collagen from the skin samples. The conventional method often involves repetitive and prolonged extraction steps to fully isolate the collagen from the skin tissue, which can take around 19 days for the collagen extraction (FIG. 8b). In addition, the extraction yield for the disclosed method may be around 70.0±7.5 wt % (based on 100 wt % of the skins), which is three times higher than the conventional method (21.3±3.6 wt %). Given the increase in collagen yield and reduction in time (much/all of which is spent at reduced temperatures (e.g. 4° C.)), the new process both reduces waste and costs.

In the method above, the acidic solvent may be aqueous acetic acid. The acid may have any suitable concentration in the aqueous media. In embodiments of the invention that may be mentioned herein, the aqueous acetic acid may have a molarity of from 0.1 to 1 M, such as from 0.3 to 0.7 M, such as about 0.5 M. The acidic solvent may be used in any suitable amount. For example, the acidic solvent may be provided in a weight to volume ratio of from 0.1:10 to 2:10, such as 1:10, where the weight refers to the weight of the pre-treated skins from a non-mammalian animal and the volume refers to the volume of the acidic solvent.

Any suitable form of blending may be used, provided that it shreds the skins into small pieces, such that a paste is formed. This blending may take place over any suitable time period. For example, the blending may be conducted over a period of from 1 to 20 minutes, such as from 2 to 10 minutes, such as about 5 minutes. The blending may make use of any suitable blender speed. For example, the blending may be conducted at from 20,000 to 50,000 rpm, such as from 30,000 to 40,000 rpm, such as about 35,000 rpm.

As the collagen within the skin of non-mammalian animals may be fragile, the method may be conducted at a temperature less than the ambient temperature of the environment. For example, the entire method may be conducted at a temperature of from 0.1 to 10° C., such as from 1 to 5° C., such as about 4° C.

While any suitable non-mammalian species may be used in the method disclosed herein, in certain embodiments that may be mentioned herein, the pre-treated skins may be bullfrog skin. For example, the bullfrogs may belong to the genus rana (e.g. the bullfrogs are of the species Rana catesbeiana).

In order to transform the paste obtained from the method above, further downstream steps may be performed. Thus, there is also provided a method of providing collagen from a non-mammalian source, the method comprising the steps of:

(aa) providing a collagen precursor mixture in the form of a paste;
(ab) diluting the paste with water and centrifuging the resulting diluted paste to provide a collagen solution and a pellet comprising pigments and collecting the collagen solution;
(ac) adding an inorganic salt to the collagen solution for a period of time (e.g. from 12 to 18 hours) to precipitate out a collagen salt, which is then collected by centrifugation;
(ad) adding an acidic solvent to the collected collagen salt to provide a free collagen mixture and subjecting the free collagen mixture to dialysis to provide a solution of collagen from a non-mammalian source.

As will be appreciated, this downstream method may be performed using the collagen precursor mixture in the form of a paste obtained from the method disclosed hereinbefore. This paste may be used as-is or, more particularly, it may be diluted. For example, the paste may be diluted by water in a ratio of from 1:10 to 1:30 vol/vol, such as from 1:10 to 1:20 vol/vol, such as about 1:10 vol/vol. Alternatively, the paste may be diluted by water in a ratio of from 1:2 to 1:10 vol/vol, such as from 1:3 to 1:7 vol/vol, such as about 1:5 vol/vol. As will be appreciated, the actual conditions may be selected by the skilled person depending on the amount of material and the apparatus used. For example, the dilution factor may be highly dependent on the volume/size of the centrifuge bottle. If the bottle has a large volume, then the particles may have to diffuse through a longer path before being deposited at the bottom of the bottle. As such, either a longer centrifuge time and/or more dilution is required when using a larger centrifuge bottle.

For the avoidance of doubt, it is explicitly contemplated that where a number of numerical ranges related to the same feature are cited herein, the end points for each range are intended to be combined in any order to provide further contemplated (and implicitly disclosed) ranges. Thus, above there is disclosed that the paste may be diluted by water in a ratio of:

from 1:2 to 1:3, from 1:2 to 1:5, from 1:2 to 1:7, from 1:2 to 1:10, from 1:2 to 1:20, from 1:2 to 1:30;
from 1:3 to 1:5, from 1:3 to 1:7, from 1:3 to 1:10, from 1:3 to 1:20, from 1:3 to 1:30;
from 1:5 to 1:7, from 1:5 to 1:10, from 1:5 to 1:20, from 1:5 to 1:30;
from 1:7 to 1:10, from 1:7 to 1:20, from 1:7 to 1:30;
from 1:10 to 1:20 vol/vol, from 1:10 to 1:30 vol/vol; or
from 1:20 to 1:30 vol/vol.

The centrifugation step in step (ab) above may be conducted at from 15,000 to 50,000×g, such as from 20,000 to 35,000×g, such as about 25,000×g. This centrifugation step may be conducted for any suitable period of time. For example, the centrifugation may be conducted fora period of from 5 to 45 minutes, such as from 10 to 30 minutes, such as about 15 minutes.

Any suitable inorganic salt may be used in step (ac) of the method above. Suitable inorganic salts include, but are not limited to sodium sulphate, ammonium sulphate, alkali metal halides (e.g. potassium chloride, sodium chloride), and combinations thereof. In particular embodiments of the invention that may be mentioned herein, the salt may be sodium chloride. Any suitable concentration of the inorganic salt may be used. For example, the inorganic salt (e.g. sodium chloride) may be provided as an aqueous solution having a concentration of from 0.5 to 4.0 M, such as from 0.5 to 1.5 M, such as about 0.9 M. Additional or alternative salting methods may be found in U.S. Pat. No. 7,964,704 (e.g. see Example 4) and https://doi.org/10.1007/BF00548873, which protocols are both incorporated herein by reference.

The centrifugation step in step (ac) above may be conducted at from 3,000 to 10,000×g, such as from 4,000 to 6,000×g, such as about 5,500×g. This centrifugation step may be conducted for any suitable period of time. For example, the centrifugation may be conducted for a period of from 5 to 45 minutes, such as from 10 to 30 minutes, such as about 15 minutes. In certain embodiments, step (ac) of the method above may only be conducted once.

The acidic solvent in step (ad) above may be aqueous acetic acid. The acid may have any suitable concentration in the aqueous media. In embodiments of the invention that may be mentioned herein, the aqueous acetic acid may have a molarity of from 0.1 to 1 M, such as from 0.3 to 0.7 M, such as about 0.5 M.

The dialysis in step (ad) above may be conducted in two rounds, where:
(i) the first round dialysis may make use of aqueous acetic acid at a concentration of from 0.01 to 0.3 M, such as from 0.05 to 0.2 M, such as about 0.1 M; and
(ii) the second round dialysis may make use of water.

As the collagen within the skin of non-mammalian animals may be fragile, the method of generating collagen from a collagen precursor mixture in the form of a paste may be conducted at a temperature less than the ambient temperature of the environment. For example, the entire method may be conducted at a temperature of from 0.1 to 10° C., such as from 1 to 5° C., such as about 4° C.

The solution of collagen from a non-mammalian source obtained in the method above may be lyophilised.

As will be appreciated, the methods above generate a precursor collagen paste and collagen, but not the desired composite scaffold material described above. Thus in a further aspect of the invention, there is disclosed a method of providing a composite scaffold material as described hereinbefore, wherein the method comprises the steps of:

(di) providing a solution of a non-mammalian collagen;
(dii) adding calcium phosphate particles and one or both of a crosslinking agent and an agent to promote self-crosslinking to the solution of a non-mammalian collagen to form a reaction mixture and allowing the reaction mixture to react for a period of time; and
(diii) removing the solvent to provide the composite scaffold material.

When present, the agent to promote self-crosslinking may be a suitable enzyme. For example, the enzyme may be a transglutaminase.

Any suitable period of time may be used to conduct the reaction. As will be appreciated, it is optimal for the period of time to be long enough to ensure that the crosslinking agent has time to fully react with the collagen. For example, the period of time may be from 12 to 48 hours, such as from 18 to 32 hours, such as about 24 hours.

Any suitable method to remove the solvent after the period of time has passed may be used. For example, the reaction mixture of step (dii) may deposited onto a flat substrate and allowed to dry to provide the composite scaffold material in the form of a film. Alternatively, the reaction mixture of step (dii) may be deposited into a mold and lyophilised to provide the composite scaffold material in the form of a three-dimensional structure.

The non-mammalian collagen in the solution of a non-mammalian collagen in step (di) above may be provided at any suitable concentration. For example, the non-mammalian collagen in the solution of a non-mammalian collagen in step (di) above may be provided at a concentration of from 5 to 20 mg/mL, such as about 10 mg/mL.

Any suitable solvent may be used in the solution of a non-mammalian collagen. For example, the solvent in the solution of a non-mammalian collagen may be aqueous acetic acid. Any suitable concentration of acetic acid may be used. For example, the aqueous acetic acid may have a molarity of from 0.1 to 1 M, such as from 0.3 to 0.7 M, such as about 0.5 M.

The calcium phosphate particles may be provided in any suitable weight:weight ratio relative to collagen. For example, the calcium phosphate particles may be provided in a weight to weight ratio relative to collagen of from 0.5:1 to 2:1, such as about 1:1.

When present, the crosslinking agent may be provided in any suitable amount relative to collagen. For example, when present, the crosslinking agent may be provided in an amount of from 3 to 15 wt %, such as from 8 to 10 wt % relative to the dry weight of the collagen.

Certain advantages may be associated with the current invention. These include, but are not limited to the following.
Non-mammalian collagen (e.g. Bullfrog Skin-Derived Collagen (BFCol) may be free from diseases, such as bovine spongiform encephalopathy (BSE), transmissible spongiform encephalopathy (TSE) and foot-and-mouth disease (FMD). Given this, non-mammalian collagens may be regarded as a safer source of collagen as compared to those from mammalian species.
Non-mammalian collagen, particularly Bullfrog skins are soft in nature and less fibrous. Without wishing to be bound by theory, it is believed that this may make the skins relatively easier to solubilise collagen from. This property may help to facilitate collagen extraction and this may help explain the ability to obtain an extraction yield of −70 wt % (relative to the weight of the skins) obtained here using the modified method of collagen extraction.
Certain non-mammalian species are commonly farmed as a food resource (e.g. Bullfrogs) and the market demand for these species is currently expanding. Given this demand, there will be a stable supply of non-mammalian skins, which translates into a stable and reliable supply of non-mammalian collagen.
As noted above, the exploitation of non-mammalian skins to obtain collagen from will reduce food waste from the supply chain.

Aspects and embodiments of the invention that may be mentioned herein include the following statements.

1. A composite scaffold comprising bullfrog skin-derived collagen, fish scale-derived hydroxyapatite (FSHA) particles and a cross-linker.
2. The composite scaffold according to Statement 1, wherein the bullfrog is of the Rana catesbeiana species or are frogs belonging to the genus rana.
3. The composite scaffold according to Statement 1 or 2, wherein the fish scales are from snakehead fish and/or elasmoid scales derived from teleosts.
4. The composite scaffold according to any one of the preceding statements, wherein the cross-linker comprises an aldehyde and/or epoxy bifunctional cross-linker such as 1,4-butanediol diglycidyl ether (BDDE).
5. Use of the composite scaffold according to any one of the preceding statements in tissue engineering.

Further aspects and embodiments of the invention will now be described by reference to the following non-limiting examples.

### EXAMPLES

### Materials

Discarded American bullfrog (Rana catesbeiana) skins and scales from snakehead (Channa micropeltes) were kindly provided by KhaiSeng Trading & Fish Farm Pte Ltd. Sodium hydroxide (NaOH), acetic acid, sodium chloride, 1,4-Butanediol diglycidyl ether (BDDE), Dulbecco's Modified Eagle's Medium/Nutrient Mixture F-12 Ham (DMEM/F-12) medium, Roswell Park Memorial Institute (RPMI)-1640 medium, sodium bicarbonate, L-glutamine, phorbol 12-myristate 13-acetate (PMA), lipopolysaccharides (LPS), paraformaldehyde (PFA), KAPA SYBR FAST (KK4618), bovine serum albumin (BSA) and Alizarin Red S were purchased from Sigma-Aldrich, USA. 10 K MWCO SnakeSkin™ dialysis tubing, antibiotic-antimycotic, Penicillin-Streptomycin, Trypsin-EDTA, Phosphate buffered saline (PBS), Bicinchoninic Acid (BCA) protein assay kit, PrestoBlue™ cell viability reagent, PureLink RNA Mini Kit (12183018A), Alexa Fluor 488 goat anti-rabbit IgG (A11034) and Hoechst 33342 nucleic acid stain were obtained from Thermo Fisher Scientific, USA. 5 mm zirconia balls and 20 μm sieve were obtained from Retsch, Germany. 10% polyacrylamide gel, Bio-Safe™ Coomassie Brilliant Blue R-250, Superscript® III First-Strand Synthesis Supermix, and Triton X-100 were purchased from Bio-Rad, USA. THP-1 monocytes and hFOB 1.19 cells were purchased from ATCC, USA. Fetal bovine serum (FBS) was obtained from Research Instruments Pte Ltd, USA. Ethanol was purchased from Merck Millipore, USA. Rabbit anti-osteocalcin (OC) antibody (ab93876) was obtained from Abcam®, USA. FSC 22 Frozen Section Media was purchased from Leica Biosystems, USA. 4′,6-Diamidino-2-phenylindole (DAPI) was purchased from Thermo Fisher Scientific, USA.

### Analytical Techniques

### Field Emission Scanning Electron Microscopy (FESEM)

Samples were sputtered with a thin layer of platinum at 20 mV for 15 s using a JEOL Auto Fine Coater (JFC-1600; JEOL Co., Japan). Following which, the samples were imaged with the JSM-7600F Schottky FESEM (JEOL Co., Japan) at an acceleration voltage of 5 kV at a magnification of ×200, ×15,000, and ×100,000.

### Attenuated Total Reflection-Fourier Transform Infra-Red (ATR-FTIR) Spectroscopy

The samples were placed directly onto the ATR sampling accessory and scanned in a range of 4000-650 cm⁻¹ at a resolution of 4 cm⁻¹ using a Frontier MIR/FIR spectrometer (Perkin Elmer Inc., USA). The infrared spectrum was captured over 32 scans, where the peaks were used to identify the chemical functional groups. Further analysis was carried out on the obtained spectra to examine the structure of the extracted collagen by measuring the peak intensity ratio between Amide III and 1450 cm⁻¹ (J. K. Wang et al., Acta Biomater. 2017, 63, 246-260; and J. K. Wang et al., J. Mater. Sci.: Mater. Med. 2016, 27, 45).

### Sodium Dodecyl Sulphate-Polyacrylamide Gel Electrophoresis (SDS-PAGE)

The lyophilized collagen was dissolved in 0.01 M acetic acid at 10 mg/mL followed by mixing with an equal volume of 1×SDS Loading Dye and heated at 95° C. for 5 min to denature the protein. Subsequently, 10 μL of the solution was loaded onto the 10% polyacrylamide gel, where the proteins were separated under a current of 0.2 A for 2 h. Thereafter, the polyacrylamide gel was stained with Bio-Safe™ Coomassie Brilliant Blue R-250 for 1 h before washing with de-staining solution for another 1 h. Finally, the pictographs of Coomassie Brilliant Blue-stained polyacrylamide gel were captured to visualize the various protein bands.

### Atomic Force Microscopy (AFM)

The topography of the collagen was examined by AFM. The collagen solution (100 μg/mL) was air-dried on a mica surface before scanning at 500 mV using a Cypher S atomic force microscope (Asylum Research, USA) under tapping mode with a scan rate of 1.0 Hz and scan size of 1.30×1.30 μm² square area.

### Energy Dispersive X-Ray (EDX) Spectroscopy

The samples were sputter-coated with a thin layer of platinum using Fine Coater JFC-1200 (JEOL Co., Japan), where the elemental spectrum was collected by area mapping at 10 kV and a magnification of ×100 using the JSM-7600F Schottky field emission scanning electron microscope (FESEM; JEOL Co., Japan). The calcium-to-phosphate ratio was then calculated based on the compositional analysis obtained from the elemental spectrum.

### X-Ray Powder Diffraction (XRD) Analysis

The XRD profile of HA was obtained by scanning the sample under theta/2theta mode using Shimadzu XRD-6000 (Shimadzu Corp., Japan) in a range of 20<20<60 with a scan speed of 1°/min and a step size of 0.05°. The XRD profile was then compared to the powder diffraction database (International Centre of Diffraction Data) to determine the chemical phases present in the HA.

### Dynamic Light Scattering (DLS) Particle Size Measurement

The particle size distribution of the HA was analyzed using a high-performance particle nanosizer (Malvern Zetasizer—Nano ZS; Malvern Instruments, UK). One milligram of the obtained powders was mixed with 100 mL of distilled water followed by sonication. Subsequently, 1 mL of the solution was transferred into a plastic disposable cuvette and placed inside the sample holder for particle size measurements.

### Statistical Analysis

All experiments were carried out in triplicate (n=3) and expressed as mean±standard deviation unless otherwise specified. The statistically significant level was analyzed using Kruskal-Wallis non-parametric one-way analysis of variance and Mann-Whitney U test, where the data was considered statistically significant with p<0.05.

### Example 1

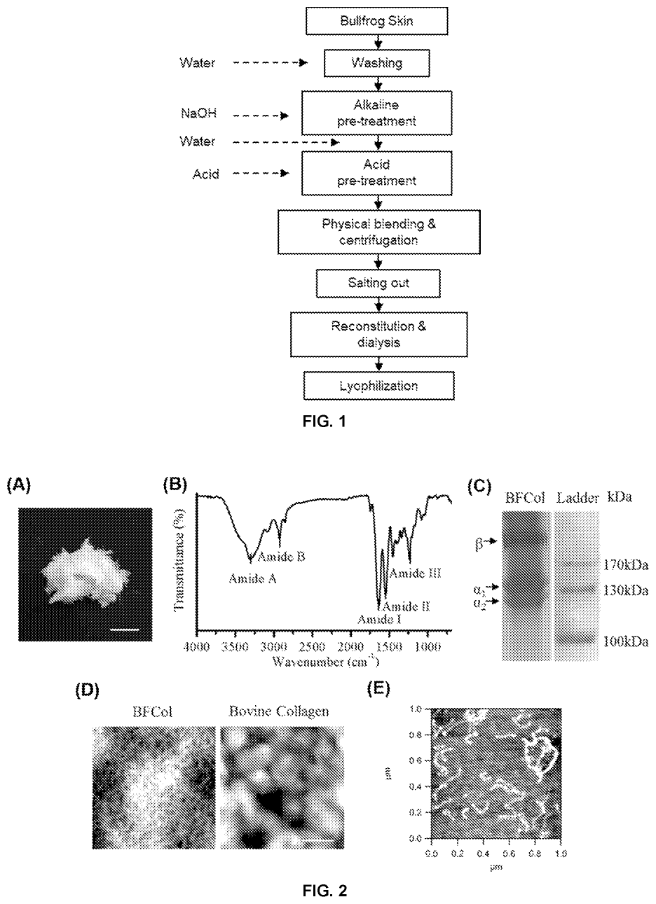

Type I tropocollagen was extracted from American bullfrog (Rana catesbeiana) skins using the in-house developed mechano-chemical method described below and in FIG. 1.

All the extraction steps were performed at 4° C. and all the solutions involved were cooled to 4° C. prior to usage. Briefly, bullfrog skins were washed with distilled water to remove any blood and impurities. Subsequently, the cleaned bullfrog skins were further treated at 4° C. with 0.5 M NaOH at a weight-to-volume ratio (wt/vol) of 1:10 over 48 h with a change of NaOH solution every 24 h to remove the unwanted protein and black pigments. The NaOH-treated bullfrog skins were then thoroughly washed with distilled water and neutralized with HCl to remove the residual NaOH. Next, the cleaned bullfrog skins were soaked in 0.5 M acetic acid at a weight-to-volume ratio (wt/vol) of 1:10 for 24 h to digest the skins, followed by blending of the skins with a PHILIPS blender at 35,000 r.p.m. for 5 min to produce a thick collagenous paste.

Subsequently, the collagen paste was further diluted with distilled water in 5 folds and centrifuged at 25,000×g for 15 min to separate the pigments from the collagen solution. The clear collagen supernatant solution was then transferred into a beaker and mixed with NaCl (0.9 M, based on the volume of the collagen supernatant solution) to induce the salting of collagen for 24 h. At the end of the salt precipitation process, the collagen salt was collected by centrifugation process at 5,500×g for 15 min. Finally, the collagen salt was reconstituted in 0.5 M acetic acid and dialyzed against 0.1 M acetic acid using SnakeSkin® dialysis tubing (10 K MWCO) for 48 h (with solution changed in between), followed by another round of dialysis in distilled water for 48 h (with solution changed in between). The final solution was subsequently lyophilized and stored at 4° C. until further usage.

### Characterization

BFCol was successfully extracted with an extraction yield of 70.0±7.5% w/w, which to the best of our knowledge, is the highest ever reported collagen extraction efficiency for frog skin (Y. Zhao et al., 3 Biotech 2018, 8, 181; J. Zhang et al., Int. J. Biol. Macromol. 2017, 101, 638-642; and H. Li et al., Food Chem. 2004, 84, 65-69). As shown in FIG. 2A, the final lyophilized BFCol takes on a whitish appearance, suggesting the successful removal of the skin pigments. ATR-FTIR spectrum of the extracted BFCol revealed typical characteristic amide peaks of collagen. ATR-FTIR spectrum of BFCol showed absorption peaks that are characteristics of amide A (˜3300 cm⁻¹), amide B (˜2920 cm⁻¹), amide I (˜1630 cm⁻¹), amide II (˜1530 cm⁻¹) and amide III (˜1200 cm⁻¹) (FIG. 2B) (J. K. Wang et al., Acta Biomater. 2017, 63, 246-260). Additionally, the absorbance peak intensity ratio of amide III band to 1450 cm⁻¹ was found to be around 1, which is indicative of the triple helix conformation in BFCol (J. K. Wang et al., Acta Biomater. 2017, 63, 246-260).

SDS-PAGE analysis was carried out and it showed that Type I collagen was successfully extracted from the bullfrog skin. SDS-PAGE analysis revealed several distinct bands near 250, 139, and 129 kDa (FIG. 2C), corresponding to the β-, α1- and α2-chains, respectively. In addition, the band width ratio of α1- and α2-chains was found to be 2:1 (FIG. 2C), similar to that of the type I collagen (J. K. Wang et al., J. Mater. Sci.: Mater. Med. 2016, 27, 45). The fibrillar structure of the extracted BFCol was significantly smaller as compared to that of commercially available bovine collagen. AFM images (FIG. 2D) show that the collagen fibrils of BFCol were approximately 38 nm in diameter, which is 10 times thinner as compared to the bovine collagen fibrils (˜374 nm). Notably, the acid soluble fraction of the BFCol existed as nanofibers with diameter of approximately 20-25 nm and length of 200-400 nm, which could correspond to small aggregates of tropocollagen (FIG. 2E) (H. Jawad et al., 5.05-Mesoscale Engineering of Collagen as a Functional Biomaterial, in: M. Moo-Young (Ed.), Comprehensive Biotechnology (Second Edition), Academic Press, Burlington, 2011, pp. 37-49; and B. D. Walterset et al., Acta Biomater. 2014, 10, 1488-1501). In general, the cell adhesion sites are in the range of 5-200 nm. Hence, compared to the typical micron-sized collagen fibrils, nano-scale BFCol could provide better support for fundamental cellular processes such as cell adhesion and proliferation, which are advantageous for tissue engineering applications (C. Y. Tay et al., Small 2011, 7, 1416-1421; and C. Y. Tay et al., Small 2011, 7, 1361-1378).

### Example 2

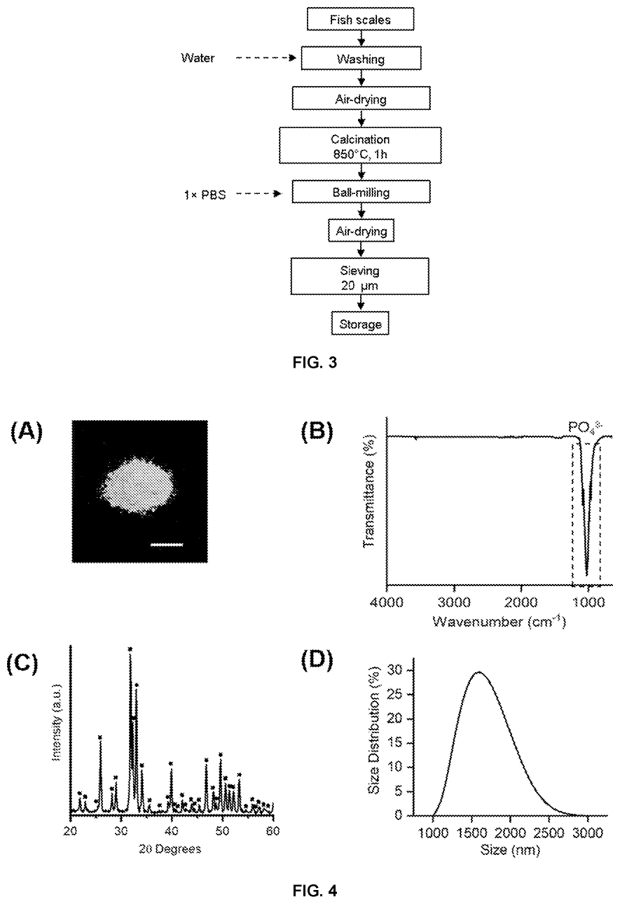

HA was harvested and processed from discarded snakehead (Channa micropeltes) fish scales via a calcination method (FIG. 3) reported previously (Y. Y. Chun et al., Macromol. Biosci. 2016, 16, 276-287) and is described below.

Freshly obtained Channa micropeltes scales were washed thoroughly with distilled water to remove any blood and impurities. Briefly, the scales were calcined in a N7/H muffle furnace (Nabertherm GmbH, Germany) at 850° C. for 1 h with a constant heating rate of 10° C./min to remove the organic matter within the fish scales. Subsequently, the heat-treated fish scales were mixed with 1×PBS at a solid-to-solution ratio (wt/vol) of 1:10, followed by ball-milling using 5 mm zirconia balls at a 10% weight-to-weight ratio of HA to zirconia balls for 24 h and air-dried for another 24 h. Thereafter, the air-dried powder was sieved through a metal mesh with 20 μm pore size and stored in a dry cabinet for further use.

### Characterization

The extraction yield of the resultant calcined product is 41.9±5.3% w/w with a calcium-to-phosphate ratio of 1.66±0.22, which is close to the stoichiometric ratio of human bones of ˜1.67 (FIG. 4A). ATR-FTIR spectrum (FIG. 4B) showed characteristic phosphate absorption peaks in the region of 1100 to 960 cm⁻¹ while the occurrence of organic peaks is visibly absent, suggesting that the sample is devoid of organic residues. Furthermore, the XRD pattern of the snakehead scales derived HA matched well with the single-phase HA from the Powder Diffraction File™ (PDF®) database (FIG. 4C). Finally, DLS analysis revealed that the HA sample has an average hydrodynamic diameter of ˜1.6 μm (FIG. 4D), which falls within the reported optimum size-range (i.e., 1-10 μm) that favors bone cell interactions and bone tissue integrations (C. Hallgren et al., Biomaterials 2003, 24, 701-710).

### Example 3

To circumvent low denaturation temperature and poor physicochemical properties of marine collagen (X. Liu et al., ACS Sustain. Chem. Eng. 2018, 6, 17142-17151), the BFCol and BFCol/HA hybrid network were structurally stabilized using BDDE to produce BDDE-crosslinked BFCol (B-BFCol) and BDDE-crosslinked BFCol/HA (B-BFCol/HA). Mechanistically, the two epoxy terminals of BDDE can react with the free carboxyl groups present in the collagen molecules to form ether bonds, while the BDDE backbone serves as the linkage between adjacent collagen chains. A one-pot synthesis method was employed to prepare B-BFCol and B-BFCol/HA hybrid biocomposite using the materials synthesized in Example 1 and 2.

### Fabrication of B-BFCol

Briefly, the lyophilized collagen (10 mg/mL) was dissolved in 0.5 M acetic acid, followed by the addition of 10% w/w BDDE crosslinker (based on the dry weight of collagen). The reaction mixture was stirred at 300 rpm for 24 h at 4° C. Thereafter, the reaction mixture was either air-dried onto coverslips cleaned with piranha solution to produce 2D coats or transferred into a mold and lyophilized to produce the 3D porous B-BFCol samples.

### B-BFCol/HA Hybrid Biocomposite

Briefly, the lyophilized collagen (12 mg/mL) was dissolved in 0.5 M acetic acid, followed by the incorporation of HA paste at a 1:1 collagen-to-HA weight ratio, together with 10% w/w BDDE crosslinker (based on the dry weight of collagen) to give a final collagen concentration of 10 mg/mL. The reaction mixture was stirred at 300 rpm for 24 h at 4° C. Thereafter, the reaction mixture was either air-dried onto coverslips cleaned with piranha solution to produce 2D coats or transferred into a mold and lyophilized to produce the 3D porous B-BFCol/HA samples.

### Characterization

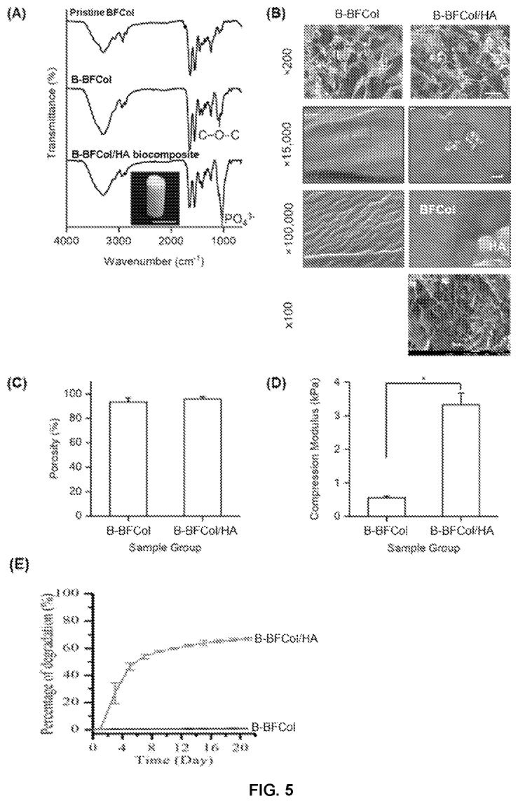

B-BFCol and B-BFCol/HA composite scaffold were successfully fabricated via lyophilization process. Successful crosslinking of the samples was first confirmed by ATR-FTIR, with the additional ether peak (C—O—C) being observed within the range of 1050 and 1250 cm⁻¹ after the crosslinking process (FIG. 5A). The presence of HA in the B-BFCol/HA hybrid biocomposite was detected by the increase in the peak intensity between 1040 and 1100 cm⁻¹, that is associated with the phosphate (PO₄³⁻) functional group. This was further confirmed by the FESEM images shown in FIG. 5B.

While both the B-BFCol and B-BFCol/HA samples displayed highly interconnected and porous architecture, the occurrence of micronized HA was limited to the B-BFCol/HA group. The average pore size of B-BFCol/HA hybrid biocomposites was found to be ˜54.6±26.3 μm, and is within the suitable pore size range (i.e. 20-1,500 μm) for tissue engineering applications (C. M. Murphy et al., Cell Adh Migr. 2010, 4, 377-381). At higher magnifications, the presence of HA micro-agglomerates against the B-BFCol nanofibrous network was clearly evident. Such hierarchical and/or multi-scale surface topographies can favor cell-material interactions and topography-induced osteogenic differentiation. As an example, a recent study by Zheng et al. has shown that the micro-nano topography promotes the cell adhesion, formation of mature focal adhesions, and osteoblast differentiation via integrin α2-PI3K-AKT signaling axis (H. Zheng et al., Front. Bioeng. Biotechnol. 2020, 8, 4630).

### Example 4

The porosity and mechanical properties of the B-BFCol/HA biocomposite samples prepared in Example 3 were evaluated, and the experimental procedures and results are provided below.

### Porosity Measurement

The porosity of the 3D hybrid biocomposites was measured using a liquid displacement method. Briefly, the individual sample was immersed in a glass bottle containing a known volume of absolute ethanol (V1) for 5 min. Subsequently, the total volume of absolute ethanol with the ethanol-impregnated sample was recorded as V2. The ethanol-impregnated sample was then removed from the glass bottle, where the residual ethanol volume was recorded as V3. The porosity of the scaffold was then estimated according to Equation 1.

$$Porosity = \frac{V 1 - V 3}{V 2 - V 3} \qquad ( 1 )$$

### Compression Modulus

The mechanical property of the 3D hybrid biocomposites was determined by measuring the compression modulus of the samples. The sample was loaded onto an Instron model 5567 universal testing machine (Instron Corp., USA) and compressed to 50% of its original height at a rate of 2 mm/min with a 10 kN load cell to obtain a stress-strain curve. The initial gradient of the stress-strain curve was then measured to give the compression modulus.

### In Vitro Degradation Study

The degradation behavior of the 3D hybrid biocomposites was studied by quantifying the amount of degraded collagen over various time points using the BCA protein assay kit. Briefly, the samples were immersed in 1×PBS and incubated at 37° C. over 21 days. At various time points, 1×PBS was collected and replaced with an equivalent volume of fresh 1×PBS. The amount of the collagen degraded was quantified using BCA Protein Assay and the cumulative degradation was plotted against time to obtain the degradation profile.

### Water Absorption of B-BFCol/HA

The water absorption capacity of the B-BFCol/HA was assessed by adding 200 μL of the cell culture medium (i.e. DMEM/F-12) in a dropwise manner, in which the time of the water being fully absorbed was being recorded.

### Results and Discussion

The porosity of both the B-BFCol and B-BFCol/HA samples were in excess of 90% (FIG. 5C). Specifically, the porosity of the B-BFCol/HA composite scaffold was approximately 95.7±2.0%. Highly porous scaffolds are attractive from the tissue engineering standpoint as they facilitate cell penetration, nutrients and metabolic waste exchange, vascularization, as well as bone formation (A. Autissier et al., Acta Biomater. 2010, 6, 3640-3648; and S. Yunoki et al., Mater. Lett. 2006, 60, 999-1002).

The mechanical properties of the scaffolds are important to ensure long-term structural and functional viability (J. Chen et al., Biophys. J. 2012, 103, 1188-1197). Notably, compared to the B-BFCol control group, the compression modulus of the B-BFCol/HA scaffold was approximately 6-fold stiffer (FIG. 5D). Therefore, the incorporation of HA successfully improved the compression modulus of B-BFCol scaffolds. Although a higher degradation profile (FIG. 5E) was observed for HA-incorporated composite scaffolds due to the embrittlement effect of HA, it is actually advantageous from the tissue engineering standpoint as it means that the scaffold is resorbable and can be replaced by the newly formed tissue eventually.

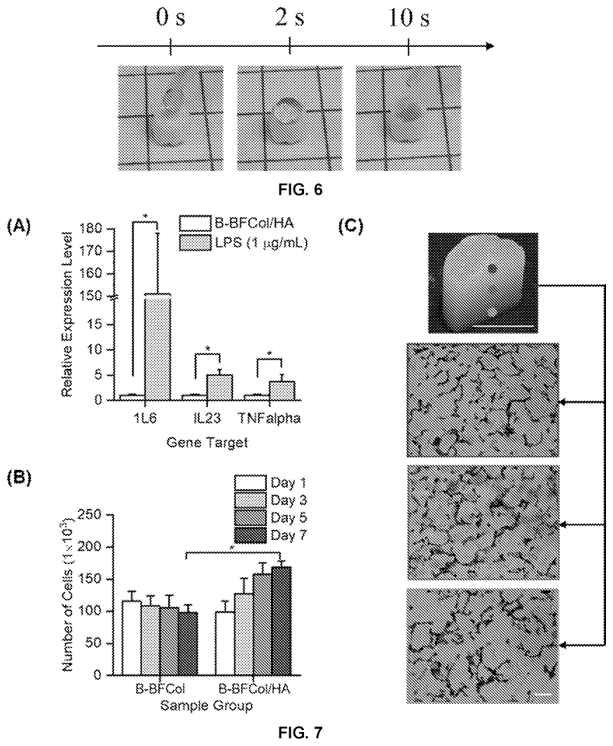

Nevertheless, the addition of micronized fish scale derived HA coupled with BDDE crosslinking significantly increased the overall stability and compressive stiffness of the B-BFCol/HA hybrid biocomposite. Specifically, the measured bulk compressive stiffness of B-BFCol/HA was estimated to be around 3.32±0.35 kPa. Although the scaffolding modulus for the formation of osteoblastic matrix is in the range of 30 kPa (N. Huebsch et al., Nat. Mater. 2010, 9, 518-526), the presence of bioactive components can induce a greater biochemical effect on the osteogenesis in which a soft substrate is amendable to support stem cell osteogenic differentiation (K. Vuornos et al., J. Biomed. Mater. Res., Part B 2020, 108, 1332-1342). Further to this, the matrix stiffness of the hybrid biocomposite can be easily enhanced by either incorporating nano-sized HA (˜6.2-fold increase in compressive modulus) instead of micron-sized HA (˜2.2-fold increase in compressive modulus) or coating the porous scaffold with HA precipitate (˜26.2-fold increase in compressive modulus) to further fine-tune the physical properties (A. J. Ryan et al., J. Anat. 2015, 227, 732-745). Additionally, the B-BFCol/HA hybrid biocomposite displayed excellent absorbency in which it was able to absorb the added solution within a few seconds, making it ideal to absorb bodily fluid and maintain a moist microenvironment supportive of tissue repair (FIG. 6). Collectively, the high porous structural fidelity, coupled with the improved mechanical properties, makes B-BFCol/HA hybrid biocomposite an ideal platform for tissue engineering applications.

### Example 5

To ensure that the extraction protocols in Examples 1 and 2, and the one-pot synthesis of B-BFCol/HA hybrid biocomposite in Example 3 are suitable for preparation of bone implants, the immunoreactivity of the B-BFCol/HA hybrid biocomposite was first examined using PMA-induced differentiated macrophages from human THP-1 monocytes. The cell culture and real-time polymerase chain reaction (RT-PCR) procedures, and experimental results are provided below.

### Cell Culture

THP-1 monocytes were cultured and expanded in RPMI-1640 medium supplemented with 10% FBS, 1.6 g/L sodium bicarbonate, and 1% Penicillin-Streptomycin under 37° C., 5% CO₂ environment, and saturated humidity. The immunoreactivity of the hybrid biocomposites was examined using PMA differentiated macrophages from human THP-1 monocytes where the pro-inflammatory genes expressed by THP-1 macrophages were examined using real-time polymerase chain reaction (RT-PCR) (J. K. Wang et al., Macromol. Rapid Commun. 2020, 41, 2000275).

### RT-PCR

Total RNA was extracted from THP-1 macrophages using a PureLink RNA Mini Kit. The concentration and quality of the total RNA were determined using NanoDrop™ 2000 (Thermo Scientific, USA). Thereafter, the synthesis of cDNA was carried out on the extracted RNA using iScript cDNA Synthesis Kit according to manufacturer's protocol where the reaction was done using a T100 Thermal Cycler (Bio-Rad, USA): Priming for 5 min at 25° C., reverse transcription for 20 min at 46° C., and reverse transcriptase inactivation for 1 min at 95° C. For the RT-PCR experiment, KAPA SYBR FAST was used where the expression levels of the target mRNA transcripts were determined using a CFX Connect RT-PCR Detection System (Bio-Rad, USA) using the following protocol: enzyme activation and DNA denaturation at 95° C. for 30 s, followed by amplification for 40 cycles with each cycle inclusive of 15 s of denaturation step at 95° C. as well as 30 s of annealing/extension and plate reading at 60° C. The threshold cycle (Cₜ) values were noted for each transcript and normalized to the internal housekeeping control. The relative quantitation of each mRNA was performed using the comparative Cₜ method as described earlier (H. Yang et al., Adv. Healthc. Mater. 2019, 8, 1900929). The listed sequence of the primers (Table 1) was obtained from primer bank (https://pga.mgh.harvard.edu/primerbank). For immunogenic response study, 1 μg/mL LPS was used as a positive control to induce polarization of resting macrophages (M0) to proinflammatory M1 phenotype.

TABLE 1
Sequences of primers and
housekeeping genes used for RT-PCR studies.

| Transcripts | Primer Sequence (5′ to 3′) |
|---|---|
| IL-6 | GTAGCCGCCCCACACAGA |
| | CATGTCTCCTTTCTCAGGGCTG |
| IL-23 | TGCAAAGGATCCACCAGGGTCTGA |
| | TAGGTGCCATCCTTGAGCTGCTGC |
| TNF-α | ATGAGCACTGAAAGCATGATC |
| | TCACAGGGCAATGATCCCAAAGTAGACCTGCCC |
| ALPL | CGTTGTCTGAGTACCAGTCCC |
| | ACCACCACGAGAGTGAACCA |
| BGLAP | CACTCCTCGCCCTATTGGC |
| | CACTCCTCGCCCTATTGGC |
| 18S | GCGGCGGAAAATAGCCTTTG |
| | ATCACACGTTCCACCTCATCC |

### Results and Discussion

In contrast to the LPS-positive control, the expression level of inflammatory mRNA transcripts such as IL-6, IL-23, and TNF-α of the B-BFCol/HA hybrid biocomposite exposed macrophage remained relatively modest, suggesting that risk of waste derived biomaterial hybrid to trigger an excessive acute inflammatory response is low (FIG. 6A).

### Example 6

The biological performance of B-BFCol/HA scaffold for bone repair was assessed with hFOB 1.19 osteoblast cells as the in vitro cell model. The cell culture and cell proliferation assay protocols, and experimental results are provided below.

### Cell Culture

hFOB 1.19 cells were cultured and expanded using DMEM/F-12 medium supplemented with 10% FBS, 1×antibiotic-antimycotic, 1.6 g/L sodium bicarbonate, and 2.5 mM L-glutamine under 34° C., 5% CO₂ environment, and saturated humidity. For cell culture studies, the samples were sterilized by ethylene oxide (EtO) gas treatment using an EOGas 4 sterilizer (Andersen Products, Inc., USA) overnight.

### Cell Proliferation Assay

The proliferation of hFOB 1.19 cells was determined using the PrestoBlue™ cell viability reagent at various pre-determined time points according to the manufacturer's recommended protocol. Briefly, the cells were seeded onto the 2D and 3D B-BFCol and B-BFCol/HA samples with a seeding density of 30 k cells/cm² and 600 k cells/mL, respectively. On pre-determined time points, the number of cells was measured by incubating the cell-seeded samples with 10% v/v PrestoBlue™ reagent for 1 h at 37° C. At the end of the incubation period, 200 μL of the solution was transferred into a 96-well plate where the fluorescence intensity (Ex 560/Em 590 nm) was measured using a SpectraMax M2 microplate reader (Molecular Devices, USA). The cell number was determined using a standard curve correlating the fluorescence intensity to the known number of cells. Further analysis was carried out to compare the population doubling rate of the hFOB 1.19 cells cultured on different samples according to Equation 2.

$$Cell doubling = \frac{ln ( {N}_{f} ) - ln ( {N}_{i} )}{ln ( 2 )} \qquad ( 2 )$$

Where N_{f} is the cell count obtained at the respective time point, and Nᵢ denotes the cell count acquired from the previous time point.

### DAPI Staining

The cell-seeded scaffold was fixed with 4% PFA overnight at 4° C., followed by soaking in FSC 22 Frozen Section Media prior to freezing at −20° C. Thereafter, the samples were cryosectioned into 15 μm thick sections using a microtome (Leica Biosystems, USA) and collected using a poly-L-lysine treated glass slides. Finally, the samples were stained with DAPI at 1 μg/mL for 5 min at room temperature in the dark, before imaged using Zeiss Axio Observer.Z1 inverted microscope (Carl Zeiss, Germany).

### Results and Discussion

As seen from FIG. 6B, hFOB 1.19 cells seeded in B-BFCol/HA 3D porous scaffold exhibited a positive proliferation profile with an increasing number of cells observed over 7 days of culture, which was not observed for the B-BFCol sample. Interestingly, not only was the rate at which the cells are proliferating significantly faster (specific doubling rate of ˜0.18 day⁻¹ for B-BFCol/HA vs −0.05 day⁻¹ for B-BFCol), the total number of cells colonizing the B-BFCol/HA scaffold was clearly higher (˜169 k for B-BFCol/HA vs ˜98 k for B-BFCol). The significant increase in cell number could be attributed to improved cell-material interactions with the HA functionalized scaffold (P. Kazimierczak et al., Int. J. Nanomed. 2019, 14, 6615) or increased extracellular matrix (ECM) mechanics that can foster cellular proliferation-promoting signal transduction such as mitogen-activated protein kinase/extracellular-signal-regulated kinase (MAPK/ERK) pathways (C. Y. Tay et al., Nanomedicine 2013, 8, 623-638).

After 7 days of culture, the cells-seeded 3D scaffolds were fixed with 4% PFA and cryo-sectioned. The cross-sectional images (FIG. 6C) show that the coverage of hFOB 1.19 cells was evidently widespread and uniformly distributed (DAPI-stained nuclei) within the B-BFCol/HA scaffold due to the high water-absorbance capacity of the hybrid biocomposite. Thus, the B-BFCol/HA scaffold could promote proper cellular activities and eventually lead to the formation of homogenous tissues during the regeneration process.

### Example 7

To evaluate the osteoinductive potential of the B-BFCol/HA biocomposite prepared in Example 3, hFOB 1.19 osteoblasts were seeded onto 2D B-BFCol and B-BFCol/HA hybrid biocomposite as described in Example 6. The expression levels of osteogenic mRNA transcript (ALPL and BGLAP) were determined via RT-PCR described in Example 5 while the protocol for immunocytochemical staining is provided below.

### Immunocytochemical Staining

The hFOB 1.19 cells seeded on 2D hybrid biocomposite samples were fixed with 4% PFA for 12 h at 4° C. on pre-determined time points. Subsequently, the cells were permeabilized with 0.2% Triton X-100 for 10 min at room temperature. The samples were then washed with 1×PBS thrice before the samples were blocked with 2% BSA solution for 1 h at room temperature. Rabbit anti-OC antibody at a dilution factor of 1:500 was added to the samples and incubated for 12 h at 4° C. Following this, the samples were washed with 1×PBS thrice and incubated with Alexa Fluor 488 goat anti-rabbit IgG (H+L) at a dilution factor of 1:500 with 0.2 μg/mL of Hoechst 33342 solution for 2 h at room temperature in the dark. The immuno-stained images were viewed and imaged with the Zeiss Axio Imager Z1 (Carl Zeiss, Germany) inverted epifluorescence microscope fitted with an Axiocam HRM camera. To facilitate cross-comparison of the samples, imaging conditions such as exposure duration, signal amplification, etc. were kept constant. Expression levels of the target proteins were processed and measured with the ImageJ freeware (https://imagej.nih.gov/ij/).

### Results and Discussion

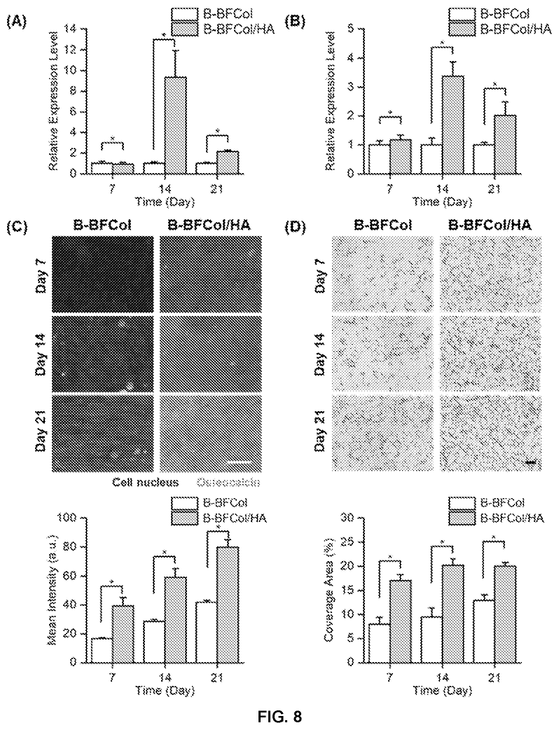

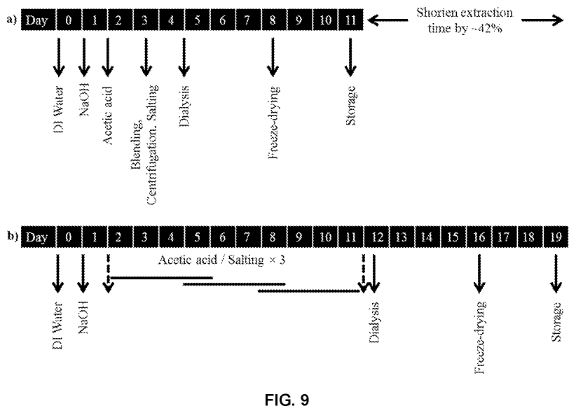

ALPL gene encodes the isoenzyme alkaline phosphatases, an early and transient marker of osteogenesis (M. Mizerska-Kowalska et al., Molecules 2019, 24, 2637). On the other hand, osteocalcin, which is encoded by the BGLAP gene is an important non-collagenous secreted factor involved in bone matrix mineralization and a late marker for bone formation (A. Rutkovskiy et al., Med. Sci. Monit. Basic Res. 2016, 22, 95). In the case of the B-BFCol/HA group, we observed a significant increase in the expression level of ALP (˜9.4 fold) relative to the B-BFCol group 14 days into the experiments (FIG. 7A). Thereafter, the upregulated state of ALP was slightly lowered (˜2.1 fold) after 21 days of culture. Similarly, BGLAP mRNA transcript (FIG. 7B) and immunocytochemical staining of OC (FIG. 7C) were consistently expressed at a higher level for B-BFCol/HA compared to the B-BFCol group by a factor of 2 to 3-fold on either day 14 or 21. These results indicate that B-BFCol/HA supported the differentiation of hFOB 1.19 into mature osteoblasts and that the inclusion of the HA component can significantly augment the scaffold's ability to induce bone differentiation events.

### Example 8

To evaluate the cell-mediated mineralization potential of B-BFCol/HA, hFOB 1.19 osteoblasts were seeded onto 3D B-BFCol (prepared in Example 1) and 3D B-BFCol/HA hybrid biocomposite (prepared in Example 3) as described in Example 6. The samples were taken for Alizarin Red S Staining Quantification assay and the assay protocol and experimental results are provided below.

### Alizarin Red S Staining

The cell-seeded 3D hybrid biocomposite samples were cryosectioned and stained with Alizarin Red S. Briefly, the cell-seeded samples were fixed using 4% PFA for 12 h at 4° C. Subsequently, the fixed samples were washed three times with 1×PBS, followed by immersion in FSC 22 Frozen Section Media and freezing at −20° C. The samples were then cryosectioned into 10 μm sections using microtome where the samples were collected and adhered onto a poly-L-lysine treated glass slides. The samples were then washed three times with 1×PBS, followed by incubation with 40 mM Alizarin red S solution for 24 h. The Alizarin Red S solution was prepared by dissolving Alizarin Red S powder in distilled water and had a final pH of 4.1, adjusted using ammonium hydroxide dissolved in water. At the end of the process, the samples were washed thoroughly with distilled water until the washes were nearly clear. Finally, the cleaned samples were imaged using a light microscope to visualize the calcium in the sections.

### Results and Discussion

After 21 days of culture period, the B-BFCol-only scaffold showed modest level of Alizarin Red S-positive mineralized deposits while the B-BFCol/HA samples clearly displayed wider coverage and deeper mineralization staining. Alizarin Red S stain quantification showed significantly increased (˜1.5 fold) staining for the B-BFCol/HA group compared to the B-BFCol-only group (FIG. 7D). This is similar to reports by others in which the presence of HA induced osteoblast mineralization as determined by an increase in Alizarin Red S stain after 10-15 days of culturing on biocomposite scaffolds (J. Venugopal et al., J. Mater. Sci.: Mater. Med. 2008, 19, 2039-2046; and J. R. Venugopal et al., Cell Biol. Int. 2011, 35, 73-80). These results are encouraging as it strongly suggests that B-BFCol/HA scaffold is able to support cell-mediated mineralization process in bone tissue development.

1. A composite scaffold material comprising:
one or both of a crosslinked polymer matrix formed from a non-mammalian collagen and a crosslinking agent and a crosslinked polymer matrix formed from a non-mammalian collagen that has undergone self-crosslinking; and
a plurality of calcium phosphate particles distributed within the crosslinked polymer matrix, wherein the composite scaffold material is porous.

2. The composite scaffold material according to claim 1, wherein the crosslinking agent is a pharmaceutically acceptable crosslinking agent.

3. The composite scaffold material according to claim 1, wherein the crosslinking agent is selected from one or more of the group consisting of genipin and compounds comprising two or more crosslinkable functional groups selected from the group consisting of amino, carboxylic acid, ester, aldehyde and epoxide functional groups.

4. The composite scaffold material according to claim 1, wherein the crosslinking agent is selected from compounds comprising two crosslinkable functional groups.

5. The composite scaffold material according to claim 1, wherein when the crosslinked polymer matrix is formed from a non-mammalian collagen that has undergone self-crosslinking, the non-mammalian collagen has been crosslinked by a transglutaminase.

6. The composite scaffold material according to claim 1, wherein, when present, the crosslinking agent forms from 3 to 15 wt % of the crosslinked polymer matrix formed from a non-mammalian collagen and a crosslinking agent.

7. The composite scaffold material according to claim 1, wherein the calcium phosphate particles have a diameter of from 10 nm to 20 μm, such as from 50 nm to 10 μm.

8. The composite scaffold material according to claim 1, wherein the calcium phosphate particles are hydroxyapatite.

9. The composite scaffold material according to claim 8, wherein the hydroxyapatite is derived from fish scales.

10. The composite scaffold material according to claim 1, wherein the non-mammalian collagen is type I collagen.

11. The composite scaffold material according to claim 1 any one of the preceding claims, wherein the non-mammalian collagen is derived from bullfrog skin.

12. The composite scaffold material according to claim 1, wherein the composite scaffold material has a porosity of from 90 to 99%.

13. The composite scaffold material according to claim 1, wherein one or more of the following apply:
(ai) the composite scaffold material has a compression modulus of from 0.5 to 4.5 kPa;
(aii) the composite scaffold material is further coated with calcium phosphate particles; and
(aiii) more than 50% of the composite scaffold material has degraded after 8 days when subjected to a 1× phosphate-buffered saline, and the degradation is measured by bicinchoninic acid (BCA) protein assay kit.

14. (canceled)

15. (canceled)

16. A method of tissue engineering comprising the step of providing a suitable amount of a composite scaffold material according to claim 1 to a subject in need thereof.

17. A method of in vitro tissue engineering, wherein the method comprises the steps of:
(bi) supplying a composite scaffold material according to claim 1;
(bii) adding cells and a suitable cellular nutrient mixture to the composite scaffold material; and
(biii) allowing the cells to grow on the composite scaffold material for a period of time.

18. The method according to claim 16, wherein the tissue engineering is bone tissue engineering.

19. A method of providing a collagen precursor mixture from a non-mammalian source, the method comprising the steps of:
(a) providing a mixture of pre-treated skins from a non-mammalian animal in an acidic solvent; and
(b) subjecting the mixture to mechanical blending to provide the collagen precursor mixture in the form of a paste.

20. (canceled)

21. A method of providing collagen from a non-mammalian source, the method comprising the steps of:
(aa) providing a collagen precursor mixture in the form of a paste;
(ab) diluting the paste with water and centrifuging the resulting diluted paste to provide a collagen solution and a pellet comprising pigments and collecting the collagen solution;
(ac) adding an inorganic salt to the collagen solution for a period of time to precipitate out a collagen salt, which is then collected by centrifugation;
(ad) adding an acidic solvent to the collected collagen salt to provide a free collagen mixture and subjecting the free collagen mixture to dialysis to provide a solution of collagen from a non-mammalian source.

22. (canceled)

23. (canceled)

24. A method of providing a composite scaffold material according to claim 1, wherein the method comprises the steps of:
(di) providing a solution of a non-mammalian collagen;
(dii) adding calcium phosphate particles and one or both of a crosslinking agent and an agent to promote self-crosslinking to the solution of a non-mammalian collagen to form a reaction mixture and allowing the reaction mixture to react for a period of time; and
(diii) removing the solvent to provide the composite scaffold material.

25. (canceled)

26. (canceled)

27. The method according to claim 17, wherein the tissue engineering is bone tissue engineering.
